(19) United States
(12) Patent Application Publication
(10) Pub. No.: US 2018/0103663 A1
(43) Pub. Date: Apr. 19, 2018

(54) RECOVERY OF INDUSTRIAL WASTE

(71) Applicant: Mohammad Ghajavand, Göteborg (SE)

(72) Inventor: Mohammad Ghajavand, Göteborg (SE)

(21) Appl. No.: 15/736,499

(22) PCT Filed: Jun. 17, 2016
(86) PCT No.: PCT/SE2016/050593

§ 371 Date: Dec. 14, 2017

(30) Foreign Application Priority Data
Jun. 18, 2015 (SE) 1550852-6

(51) Int. Cl. A23L 7/104; C12P 1/04

(52) U.S. Cl. CPC A23L 7/104; C12P 1/04; A23Y 2220/39; A23Y 2220/47; A23Y 2220/03

(57) ABSTRACT

There is provided a method for recovering spent grain, comprising the steps of: a) adding a batch of mixed solution of a batch of spent grain and a first fermenting composition to a second fermenting composition; b) mixing the added batch of mixed solution with the second fermenting composition, whereby a fermented mixture is obtained; and c) separating the fermented mixture into a liquid product and a solid product; wherein the liquid product and the solid product have a prolonged durability compared to the durability of the batch of spent grain when mixed solution has been added in step a). An advantage of the inventive method is that by-products such as spent grain are efficiently turned into valuable products.

### FIELD OF THE INVENTION

The present invention relates to the field of recovering industrial waste. More specifically, the present invention relates to a method for recovering spent grain. The present invention further relates to a liquid product and a solid product obtainable by the method for recovering spent grain. The present invention further relates to use of such liquid or solid product as an ingredient in human food.

### BACKGROUND OF THE INVENTION

Today there is a high awareness of the importance of achieving sustainable industrial cycles as a part of minimizing some of the negative impact that humans have on the environment. Every year huge amounts of beer is produced worldwide. The main by-product derived from the brewing industry is spent grain. Historically the brewing industry has discharged spent grain directly into the nature. Such discharge may however lead to fresh water pollution. Research on alternative ways of disposing spent grain has been examined It has further been researched on alternative uses of spent grain, since it is very nutritious and rich in proteins and dietary fibres. It has been suggested that spent grain may be used as e.g. animal feed, biogas production or as combustion material.

One drawback of spent grain is that it comprises a high amount of water, such as about 70 to 80% by volume. It may therefore be costly and counterproductive to dry spent grain in order to use it as e.g. combustion material. Another drawback of spent grain, which makes it difficult to handle and process, is that it starts to turn rancid soon after it has been discharged from the brewery. It is therefore necessary to store it in refrigerators, which is energy consuming.

No efficient methods exist today for recovering spent grain. There is therefore a need in the art for improved methods which allow for efficient recovery of spent grain.

### SUMMARY OF THE INVENTION

Objects of the present invention are to provide improved methods which allow for efficient recovery of spent grain, by which the entire spent grain may be recovered.

These objects are achieved, in a first aspect, by means of a method for recovering spent grain, comprising the steps of:
a) adding a batch of mixed solution of a batch of spent grain and a first fermenting composition to a second fermenting composition;
b) mixing the added batch of mixed solution with the second fermenting composition, whereby a fermented mixture is obtained; and
c) separating the fermented mixture into a liquid product and a solid product; wherein the liquid product and the solid product have a prolonged durability compared to the durability of the batch of spent grain when mixed solution has been added in step a).

The inventive method thus provides a more efficient method of refining a batch of spent grain derived from the beer brewery into valuable products having prolonged durability compared to the batch of spent grain. The products further have a prolonged durability compared to the batch of mixed solution. The prolonged durability is e.g. achieved by the fermentation process performed by the fermenting bacteria comprised in the fermenting composition. The mixed solution is mixed with a second fermenting composition in order to increase its degree of fermentation. This will further be described below. Since the inventive method contributes to useful products in the form of a liquid product and a solid product, the method contributes to a more sustainable industrial cycle in the beer industry wherein the produced waste at the beer brewery may be decreased.

The inventive method provides an efficient method in which the entire spent grain may be recovered, wherein the produced liquid product may be used in the human food and/or cosmetic industry and the solid product may be used in the human food industry. The liquid product may e.g. be a part of or constitute the liquid ingredient for making dough products or may be a basic ingredient in nutritious soft drinks. The liquid product comprises many minerals and healthy amino acids. The liquid product may further be an ingredient in e.g. creams or lotions, mouth rinsing agents, or throat washing agents. The solid product is rich in protein and dietary fibres and may be a basic ingredient in e.g. bakery, dairy and/or fast food industry. Both the liquid product and solid product have a probiotic effect which when used in e.g. food products, enhance the probiotic and durability properties of the food products.

The liquid product may further be reused at various steps of the inventive method, thereby contributing to a more continuous method of recovery. The method not only contributes to a more environmental industrial process, but the method of recovering spent grain also enable spent grain of being a source of income and benefit to the brewery which further promotes its recovery. The inventive method further contributes to a cost and energy effective process of making use of spent grain.

The method steps a) to c) of the inventive method as described above may be considered to constitute one cycle of the inventive method.

In an embodiment, the method prior to step a) comprises the steps of:
i. adding the batch of spent grain to the first fermenting composition; and
ii. mixing the added batch of spent grain with the first fermenting composition, whereby the mixed solution is obtained.

Since the batch of spent grain may e.g. be added to the fermenting bacteria at a very high temperature, the conditions for the fermenting bacteria may hence not be optimal. Due to e.g. the high temperature some of the fermenting bacteria may die. Therefore it is important to add and mix the mixed solution with the second fermenting composition. The second fermenting composition may increase the degree of fermentation, whereby the fermented mixture may have a higher degree of fermentation compared to the mixed solution. The process steps i. and ii. may refer to initial steps which may be performed on the batch of spent grain at the beer brewery in order to improve its durability. When spent grain is released as by-product from the beer brewery it is normally sterile due to it having a temperature of about 100° C. Spent grain may start to turn rancid at temperatures of about below 45° C. This embodiment provides efficient method steps which may hinder or at least slow down the process in which spent grain turn rancid after its release as by-product from the beer brewery. The batch of spent grain may directly after it has been released from the beer brewery be added and mixed with the first fermenting composition. Hence there may be no or very little production of e.g. unwanted microorganisms within the batch of spent grain. The mixed solution may therewith have an improved durability in view of the batch of spent grain. The mixed solution may thereby e.g. be stored at the beer brewery or at an intermediate storing facility, or may be transported to a processing facility at ambient temperatures without turning rancid, which alleviates the need of refrigerating the mixed solution which is energy consuming. The mixed solution may be durable for up to a week before it needs to be further processed.

In an embodiment, the batch of mixed solution is a first batch of mixed solution, wherein a portion of the fermented mixture obtained in step b) is reused in the method comprising the steps of:
a) adding a second batch of mixed solution to the portion of fermented mixture;
b) mixing the added second batch of mixed solution with the portion of fermented mixture, whereby a second fermented mixture is obtained; and
c) separating the second fermented mixture into a second liquid product and a second solid product.

This embodiment provides a continuous process wherein the fermented mixture obtained in step b) in the first cycle of the inventive method may be reused in a subsequent cycle. Typically, a portion of the fermented mixture may be used to start the fermentation process of a subsequent batch of spent grain in a subsequent cycle of the inventive process. This embodiment may provide the advantage of not having to provide a new fermenting composition after every cycle of recovering spent grain. Moreover, further generations of fermenting bacteria may be more active and/or durable in a subsequent batch of fermenting composition, liquid product or a portion of fermented mixture.

In an embodiment, the batch of mixed solution is a first batch of mixed solution, wherein the liquid product obtained in step c) is reused in the method comprising the steps of:
a) adding a third batch of mixed solution to the liquid product;
b) mixing the added third batch of mixed solution with the liquid product, whereby a third fermented mixture is obtained; and
c) separating the third fermented mixture into a third liquid product and a third solid product.

In this embodiment, method steps are provided in which the obtained liquid product may be reused in the inventive method. The liquid product may be reused e.g. to ferment a subsequent batch of mixed solution in a subsequent cycle of the inventive method. The liquid product may further be reused in the initial steps of the inventive method by adding and mixing the liquid product with a batch of spent grain obtained at the beer brewery.

In an embodiment, water is added in step a) to the second fermenting composition; or water is added in step a) to the portion of fermented mixture; or water is added in step a) to the liquid product.

By adding water to the second fermenting composition or the portion of fermented mixture or the liquid product more volume of at least the liquid product may be obtained. The second fermenting composition or the portion of fermented mixture or the liquid product may hence last longer. It does not matter in which order the water, batch of spent grain and fermenting composition, respectively, are added. 25 to 35% water in relation to the total amount of volume may be added, such as about 30% water. It should be understood that water may also be added to any subsequent fermenting compositions or subsequent portions of fermenting mixture or subsequent liquid products.

In an embodiment, the method further comprises the steps of:
d) wet grinding the solid product, the second solid product and/or the third solid product, and optionally
e) vacuum packaging the wet ground solid product, the wet ground second solid product and/or the wet ground third solid product.

It may be an advantage of wet grinding the solid product since the wet ground product may be more suitable for use in the food industry, such as in bread dough, vegetarian sausage or meat balls. In the process of wet grinding, the hulls from e.g. rye and/or barley derived from spent grain may be wet ground into smaller parts. The wet ground solid product may be used in larger amounts within food products compared to the solid product. The vacuum packaged wet ground solid product may be stored in cool or refrigerated temperatures for up to 3 months without turning rancid.

In an embodiment, step d) further comprises spray-drying the wet ground solid product, the wet ground second solid product and/or the wet ground third solid product. The step of additionally spray drying the wet ground product leads to a product comprising a decreased amount of moisture as compared to the wet ground product.

In an embodiment, the second fermenting composition, the portion of fermented mixture or the liquid product being 5 to 50% of the total volume, such as 25 to 35% of the total volume.

In an embodiment, the method prior to step i) initially comprises adjusting the pH of the first fermenting composition to a pH below 4.5, such as a pH in the range of from 3.5 to 4.5. The pH of the portion of the fermented mixture and the liquid product may have a pH of below 4.5, such as in the range of from 3.5 to 4.5. At this pH most of the carbohydrates within the batch of spent grain have been converted into amino acids. The pH of the fermented mixture may hence be tested as a measurement if the same is fully fermented and thus ready for further processing, e.g. the step c) of separation.

In an embodiment, the separation in step c) is performed by filtration or centrifugation. It should however be understood that any other means of separation known in the industry may also be employed in step c).

In an embodiment, the mixing in step b) is conducted at a temperature in the range of from 5 to 50° C., such as in the range of from 20 to 45° C., e.g. in the range of from 37 to 40° C. The most effective fermentation may be performed at a temperature within the range of from 37 to 40° C.

In an embodiment, the mixing in step b) is performed within a time range of from 4 to 24 hours. The time duration of the mixing may be dependent on the amount fermenting composition, portion of fermented mixture or liquid product used, i.e. a higher amount of the same may lead to a faster fermentation process.

In an embodiment, the first and/or second fermenting composition comprising fermenting bacteria being lactic acid bacteria, such as lactic acid bacteria chosen from the group consisting of Lactobacillus helveticus 14492, Lactobaciullus kefir 14502, and Lactobacillus acidophilus 14499, or any combinations thereof. Consequently, also the portion of fermented mixture and the liquid product comprises the above named fermenting bacteria. The skilled person in the art would realise that also other lactic acid bacteria may be used.

There is, in a second aspect, provided a liquid product obtainable by the method according to the first aspect of the present invention. The previously stated advantages of the method also apply to the liquid product obtainable by this method.

There is, in a third aspect, provided a solid product obtainable by the method according to the first aspect of the present invention. The previously stated advantages of the method also apply to the solid product obtainable by this method.

In a fourth aspect of the invention, there is provided a use of the liquid product according to the second aspect as an ingredient in human food.

In a fifth aspect of the invention, there is provided a use of the solid product according to the third aspect as an ingredient in human food.

The uses according to the fourth and fifth aspects are advantageous as the protein content of the liquid and solid products are high. Furthermore, when carrying out the method for obtaining said liquid and solid products, a large amount of carbohydrates will be converted to amino acids, thus resulting in products low in carbohydrates and high in protein contents. The liquid and solid products may advantageously be used as an ingredient in various human food stuffs, including as an ingredient in the dairy industry (including making ice cream), in bakeries, in fast-food production, etc.

As explained elsewhere in this disclosure, the inventive method allows the spent grain to be taken directly at, for instance, a brewery, and mix it with a first fermenting composition. Spent grain is normally sterile when it comes from the brewery and it important to add the first fermenting composition as soon as possible while the spent grain is still sterile (before it starts to turn rancid and/or oxidized). Because of the quick/direct mixing, the spent grain is maintained sterilized. It will not have time to turn rancid or become oxidized. The spent grain from, for example, beer production has a high temperature (approximately 100° C.), but the temperature quickly drops and at approximately 45° C. it would start to turn rancid. The fermentation prevents this from happening. However, this means that the conditions for the fermenting bacteria may not be optimal, and that some fermenting bacteria die at high temperatures. To obtain a well fermented and protein rich product, the inventive method also includes a second fermenting composition. The two fermenting compositions do not need to be used at the same location. On the contrary, the inventive method allows a decoupling of these fermenting steps. After the spent grain has been mixed with the first fermenting composition, the sterilized spent grain can then be stored and transported without urgency to a completely different production facility, where the fermented solution is mixed with a second fermenting composition. After separation, the solid product may be used as a protein rich ingredient in human food, while the liquid product may be used as a protein rich ingredient in human food or as cosmetics or reused as a first fermenting composition and sent back to the brewery for mixing with a new batch of spent grain. The use of two fermenting compositions not only enables the above-mentioned decoupling of production facilities and products of long durability, but also results in the protein content becoming higher than if a single fermenting composition would have been used.

According to at least some exemplary embodiments of the present invention, the method comprises three fermenting stages. The first stage includes providing a first fermenting composition. This may be a liquid which is fermented to obtain a first batch of a first fermenting composition, but the first fermenting composition could instead or also be recovered as the liquid product (or part of the liquid product) that has been separated from the solid product (i.e. after step c of the inventive method). Bacteria are in a latent state, and the first fermenting composition can be kept in a sealed container even for months for later use. The second stage includes adding the spent grain to the first fermenting composition. This will give the bacteria access to new carbohydrates. The bacteria will become active and the fermentation process is started again. Some of the bacteria may die due to the high temperature of the spent grain (therefore it is difficult to achieve controlled fermentation at this stage). The third stage includes the final and controlled fermentation when mixing with the second fermenting composition. Bacteria convert carbohydrates to amino acids to a certain level depending on the desired nutrition contents of the end product.

### BRIEF DESCRIPTION OF THE DRAWINGS

Referring now to the Figures, which are exemplary embodiments, wherein:

FIG. 1 is a flow chart depicting one example of a method for recovering spent grain according to the present invention;

FIG. 2 is a schematic representation depicting an example of a system, for carrying out the initial steps of the inventive method, for recovering spent grain.

FIG. 3 is a schematic representation depicting an example of a system, for carrying out the inventive method, for recovering spent grain.

FIG. 4 is a schematic representation depicting an alternative example of a system, for carrying out the inventive method, for recovering spent grain.

### DETAILED DESCRIPTION

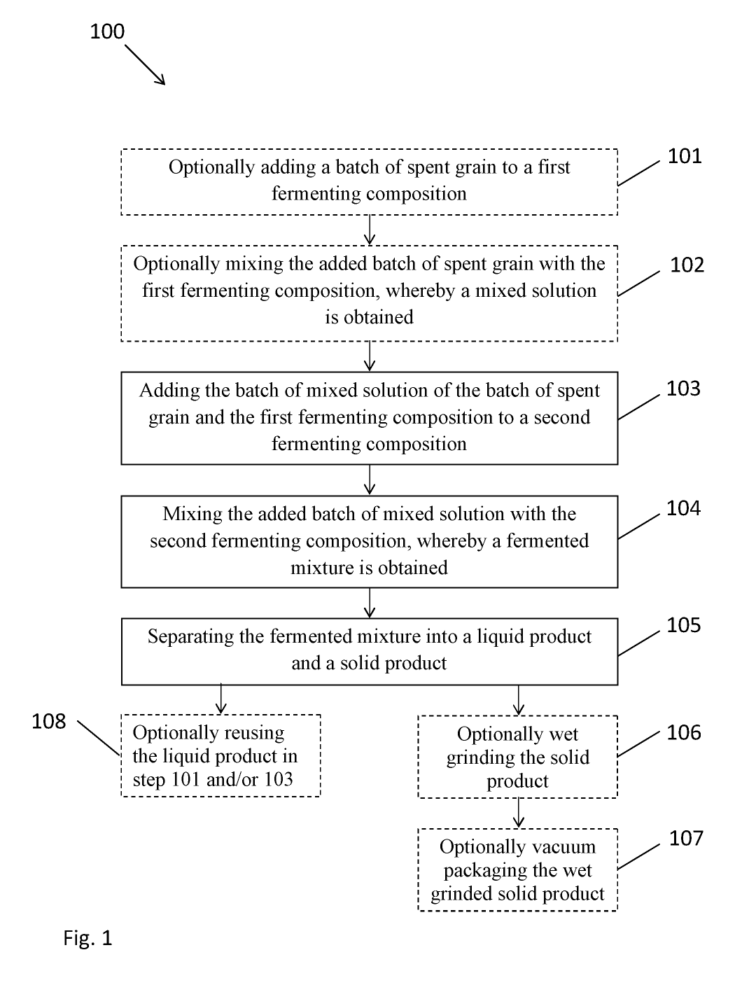

In order to reduce our impact on the environment and to aim towards a more sustainable society, it is important to recycle and make use of e.g. industrial waste. The inventive method concerns the transformation of industrial waste, such as spent grain from beer breweries, into valuable products e.g. to be used within the food industry and/or cosmetic industry. A method for achieving this is shown in FIG. 1.

FIG. 1 is a flow chart of a method 100 for recovering spent grain according to one embodiment of the present invention. In a first step 103, a batch of mixed solution of a batch of spent grain and a first fermenting composition is added to a second fermenting composition. Thereafter, the added batch of mixed solution is, in a further step 104, mixed with the second fermenting composition, whereby a fermented mixture is obtained. The steps of adding 103 and mixing 104 may for example be carried out concurrently or as separate consecutive steps. Step 104 is followed by step 105 of separating the fermented mixture into a liquid product and a solid product. The liquid product and the solid product, respectively, have a prolonged durability compared to the durability of the batch of spent grain obtained as by-product from the beer brewery when mixed solution has been added in step 103.

With regard to spent grain, the first step 103 may be preceded by optional steps 101 and 102. These steps are optional since they may be performed by the inventor or they may be performed by the personnel of the brewery and subsequently bought by the inventor. The optional step 101 is performed by adding the batch of spent grain to the first fermenting composition. Thereafter the added batch of spent grain is, in the further optional step 102, mixed with the first fermenting composition, whereby the mixed solution is obtained. The optional steps of adding 101 and mixing 102 may for example be carried out concurrently or as separate consecutive steps. The optional step 101 may as well be performed by adding the first fermenting composition 21 to the batch of spent grain 23. The steps 101 and 102 are typically performed at the beer brewery on the batch of spent grain directly after it has been released as a waste stream from the process of brewing beer.

The step 105 may be followed by the optional steps 106 to 108. These steps are optional since the liquid product and the solid products may be sold as they are or may be further processed by the inventor. The solid product may in an optional step 106 be wet ground and in a subsequent optional step 107 be vacuum packaged. The liquid product may in an optional step 108 be reused as the first fermenting composition in the optional step 101 or as the second fermenting composition in step 103.

The term “spent grain” should herein be understood as a by-product, which is derived from the beer industry, such as directly from a process of brewing beer. Spent grain may be used interchangeable with e.g. “draff” or “Brewer's grain”. The spent grain is rich in dietary fibres and proteins and it comprises about 70 to 80% of water by volume. The spent grain is typically sterile when it is released from the beer brewery since it has a temperature of about 100° C. Spent grain starts to decompose at temperatures of below about 45° C. and has a low durability at room temperature. A batch of spent grain may be a predefined amount of spent grain, i.e. a predefined amount of by-product from the beer brewery. The batch of spent grain may e.g. be added to a transportation tank comprising a first fermenting composition.

The term “first fermenting composition” should herein be understood as a composition comprising fermenting bacteria, which is capable of fermenting spent grain. The process of fermentation is well known in the art and will not be explained in-depth herein. The fermenting process has an enhancing and prolonging effect on durability of the product being fermented. The term “second fermenting composition” should herein be understood as being a composition comprising fermenting bacteria, which is capable of fermenting spent grain. The second fermenting composition may have a similar composition as the first fermenting composition, which is comprised in the transportation tank in the optional step 101 and mixed with the added batch of spent grain in the optional step 102 at the beer brewery. The second fermenting composition may originate from the same fermenting batch as the first fermenting composition or from a further batch. This also applies to any further fermenting compositions.

The term “mixed solution” should herein be understood as an intermediate product obtained by mixing at least two components, e.g. by mixing a solution of a batch of spent grain with a first fermenting composition. The mixed solution is at least partially fermented by fermenting bacteria comprised in the first fermenting composition. The mixed solution may have a prolonged durability compared to the durability of spent grain. The mixed solution may be stored at ambient temperature, such as in a time period of up to a week. Before this time period runs out, the mixed solution should be further processed in order for the mixed solution not to turn rancid. A batch of mixed solution may be a predefined amount of mixed solution. The batch of mixed solution may be added to a processing tank comprising a second fermenting composition. The processing tank is typically placed at a processing factory for processing the batch of mixed solution into a liquid product and a solid product. The term “second batch of mixed solution” should herein be understood as a solution which is similar to the mixed solution in terms of what it comprises. The batch of second mixed solution may be obtained from a second cycle of the optional initial steps of the inventive method. It may be added to the processing tank subsequent to the separation step of the fermented mixture.

The term “fermented mixture” should herein be understood as a second intermediate product obtained by mixing at least two components, e.g. by mixing the batch of mixed solution with the second fermenting composition. The fermented mixture is typically obtained under controlled process parameters for allowing the fermentation process to occur within the mixture. The fermented mixture is typically fermented to a higher degree of fermentation compared to the mixed solution.

The term “liquid product” should herein be understood as a product which has a water/moisture content of more than 90% and a solid content of less than 10%. The liquid product comprises fermenting bacteria.

The term “solid product” should herein be understood as a product which has a water/moisture content of less than 40% and a solid content of at least 60%.

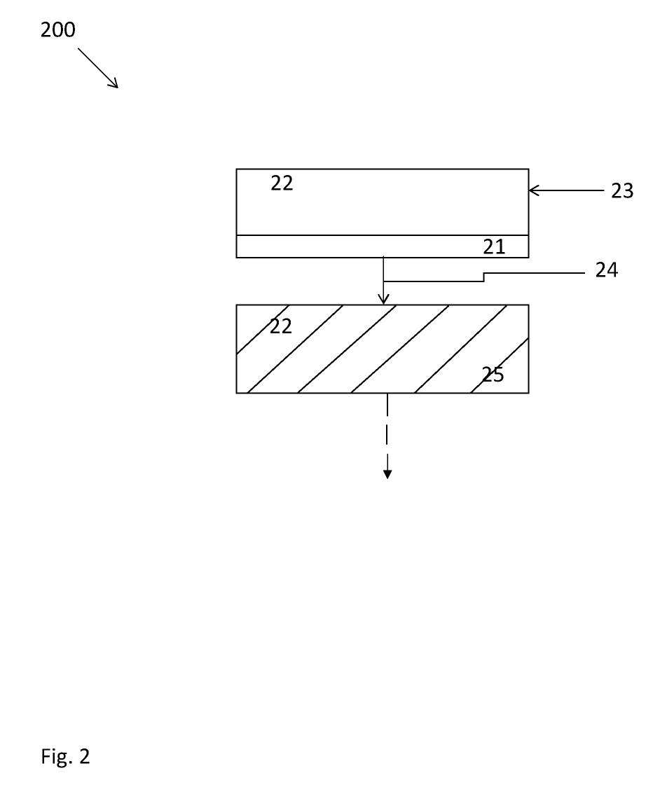

FIG. 2 is a schematic representation depicting a system 200 for carrying out the optional initial steps 101, 102 of the inventive method. The optional initial steps typically take place at the beer brewery. A transportation tank 22, such as a stainless transportation tank, being prefilled with a first fermenting composition 21, is transported to the beer brewery. Alternatively, the first fermenting composition 21 is added in an initial step to the transportation tank 22 at the beer brewery. The first fermenting composition constitute in the range of from 5 to 50% of the total volume of the transportation tank, such as about 25 to 35% of the total volume. At the beer brewery a batch of spent grain 23 is added to the transportation tank 22 comprising the first fermenting composition 21. The added batch of spent grain constitutes the remaining volume in the transportation tank 22, such as in the range of from 50 to 95% of the total volume of the transportation tank 22, such as in the range of from 65 to 75% of the total volume. Alternatively the first fermenting composition is added to the transportation tank already comprising the batch of spent grain. The percentages described above may apply to the total volume of first fermenting composition and the added batch of spent grain, for example when the transportation tank is not fully filled. The transportation tank 22 may comprise a built-in mixer which slowly mixes 24 the added batch of spent grain 23 with the first fermenting composition 21, thereby providing a mixed solution 25 within the transportation tank 22. The mixed solution 25 may thereafter be placed in intermediate storage at the beer brewery or transported to an intermediate storage facility or directly to a processing factory for further processing.

Alternatively, the batch of spent grain 23 may be added as one or more sub-batches of spent grain at different occasions during one day or during a week.

The mixing facilitates the fermentation process performed by the fermenting bacteria throughout the volume of the transportation tank 22. The mixed solution may be partially fermented. The degree of fermentation within the mixed solution is dependent on e.g. the amount of active fermenting bacteria therein. Since the batch of spent grain may e.g. be added to the fermenting bacteria at a very high temperature, the conditions for the fermenting bacteria may hence not be optimal. Due to the high temperature some of the fermenting bacteria may die. Moreover, the temperature which prevails at intermediate storage at e.g. the beer brewery or during transportation from the beer brewery to the processing factory of the transportation tank comprising the mixed solution, may also affect the amount of active fermenting bacteria within the mixed solution. The degree of fermentation within the mixed solution is however high enough for achieving a prolonged durability of the mixed solution 25 compared to the batch of spent grain 23. Hence, the mixed solution does not turn rancid at ambient temperatures, and it is protected against oxidation and harmful microorganisms, which may otherwise thrive in the batch of spent grain. The high amount of enzymes within the first fermenting composition 21 may also contribute to the enhanced durability of the mixed solution.

Thanks to e.g. the fermentation process the mixed solution may be stored for up to a week before it has to be processed further e.g. in order to not turn rancid. Moreover, the mixed solution may be stored or transported at ambient temperatures with maintained durability effect.

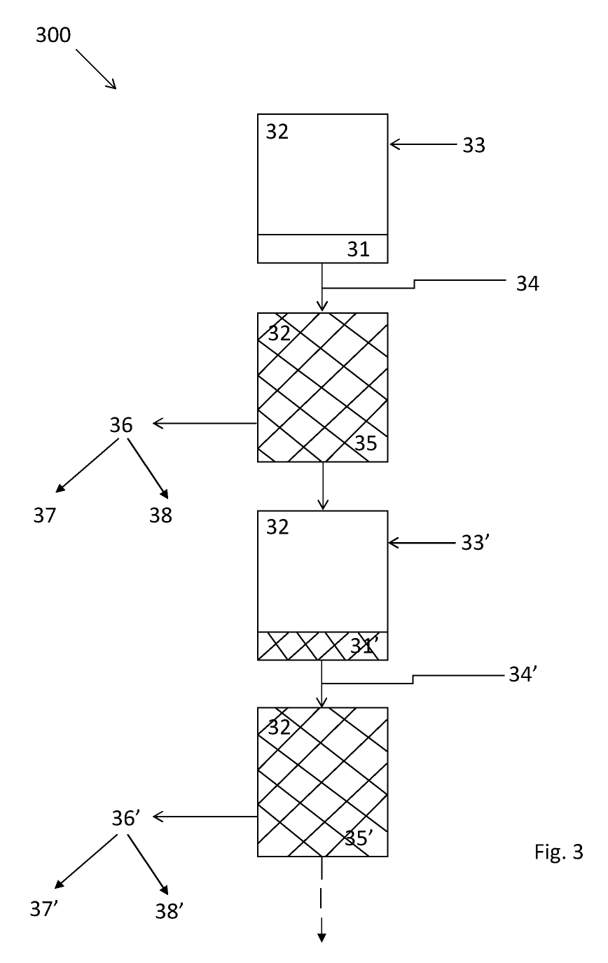

FIG. 3 is a schematic representation depicting a system 300 for carrying out the inventive method. These method steps typically take place at the processing factory for further processing of the mixed solution. The mixed solution 25 obtained in the optional initial steps of the inventive method, may be delivered to the processing factory in its transportation tank 22. Initially, a second fermenting composition 31 is added to a processing tank 32 at the processing factory. The second fermenting composition 31 may constitute in the range of from 5 to 50% of the total volume of the processing tank 32, such as about 25 to 35% of the total volume. A batch of the mixed solution 33 is added to the processing tank 32 comprising the second fermenting composition 31. The added batch of mixed solution 33 may constitute the remaining volume in the processing tank 32, such as in the range of from 50 to 95% of the total volume of the transportation tank 32, such as in the range of from 65 to 75% of the total volume. The batch of mixed solution 33 is mixed 34 with the second fermenting composition 31, whereby a fermented mixture 35 is obtained. Alternatively, the batch of spent grain 33 is added to an empty processing tank followed by adding the second fermenting composition 31 to the processing tank 32 comprising the added batch of spent grain 33. The processing tank 32 may comprise a mixer for performing the mixing. The mixing may be performed under controlled processing parameters, such as at a temperature within the range of from 37 to 42° C., and/or with a slow mixing with a duration of about 5 minutes every second hour. The mixing 34 facilitates the fermentation process performed by the fermenting bacteria throughout the volume of the processing tank 32. The fermented mixture 35 is typically fully fermented after a time duration of within the range of from 4 hours to 24 hours. The time duration may depend on the amount of fermenting bacteria present in the mixture of mixed solution 33 and second fermenting bacteria 31. The time duration may further depend on the relative amount of second fermenting composition 31 in relation to the total volume of the mixture. Generally, the higher the relative amount of first fermenting composition and the higher the relative amount of second fermenting composition, the faster is a fully fermented mixture obtained.

After the mixing step 34, the fermented mixture is transferred away from the processing tank 32 and separated 36 into a liquid product 37 and a solid product 38. The step of separation 36 may be performed by e.g. filtration or centrifugation. The liquid product 37 and the solid product 38 have a prolonged durability compared to the batch of spent grain 23 derived from the beer brewery. The liquid product 37 and the solid product 38 may further have a prolonged durability compared to the mixed solution 25 derived from the initial steps of the inventive method. The enhanced durability of the liquid product 37 and the solid product 38 may be due to its enhanced degree of fermentation. These method steps may be considered to constitute one cycle of the inventive method.

Not all of the fermented mixture 35 may be transferred to the separation step 36, but a portion of the fermented mixture 31′ may be left within the processing tank for fermenting a second batch of mixed solution 33′. The portion of fermented mixture may constitute in the range of from 5 to 50% of the total volume of the processing tank 32, such as about 25 to 35% of the total volume. The amount of portion of fermented mixture which is left in the transportation tank may determine the speed of fermentation. Hence, in a second cycle of the inventive method, the portion of fermented mixture 31′ may be reused for fermenting a second batch of mixed solution 33′. The second batch of mixed solution 33′ may be obtained from the initial steps at the beer brewery. It may originate from the same batch as the first batch of mixed solution or a further batch of mixed solution. The second batch of mixed solution 33′ is added to the portion of fermented mixture 31′ in the processing tank 32. Thereafter the added second batch of spent grain 33′ is mixed with the portion of fermenting mixture 31′, whereby a second fermented mixture 35′ is obtained. Thereafter, the second fermented mixture 35′ is separated into a second liquid product 37′ and a second solid product 38′. These method steps may be considered to constitute a second cycle of the inventive method.

Not all of the second fermented mixture 35′ may be transferred to the separation step 36′, but a second portion of the second fermented mixture 35′ may be left within the processing tank 32 for fermenting a third batch of mixed solution. Any third or further cycles of the inventive method may thereafter repeat the described method steps of adding, mixing and separating in order to produce further liquid and solid products.

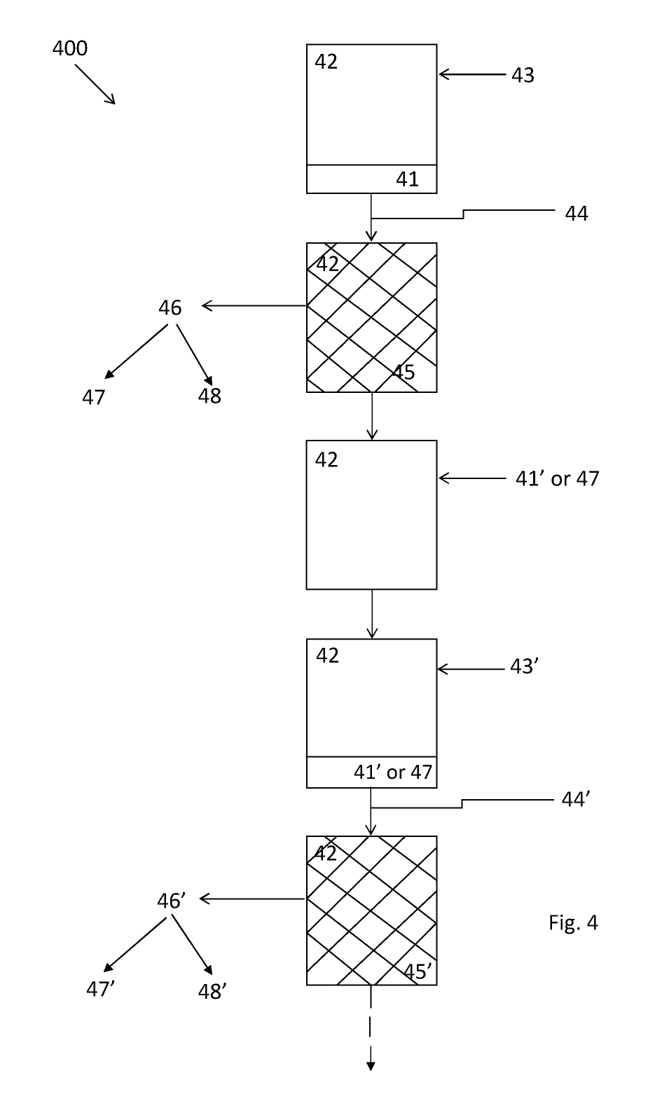

Water may be added to the processing tank 32 at the beginning of each cycle, such as prior to or subsequent to adding the batch of mixed solution 33, 33′. Alternatively, water may be added prior to adding the fermenting composition 31 to the processing tank 32. Water may constitute in the range of from 25 to 35% in relation to the total volume, such as about 30%. In an example, a batch of spent grain may constitute 20 to 40%, e.g. about 30%, and water and fermenting composition may constitute about 60 to 80%, e.g. about 30% water and about 40% fermenting composition, of the total volume of the mixture. An alternative embodiment is shown in FIG. 4, which is a schematic representation depicting a system 400 for carrying out the inventive method. These method steps typically take place at the processing factory for further processing of the mixed solution. The first cycle of the method of adding the batch of the mixed solution 43 to the processing tank 42 comprising the second fermenting composition 41, mixing the added batch of mixed solution 43 with the second fermenting composition 41, whereby the fermented mixture 45 is obtained, and separating the fermented mixture 45 into a liquid product 47 and a solid product 48, are performed in the same way as described in relation to FIG. 3. However, the entire fermented mixture 45 obtained after the mixing step 44 may be transferred to the separation step 46, leaving the processing tank empty. Thereafter, a third fermenting composition 41′ or the liquid product 47, which was obtained in the separation step 46 in the first cycle, may be added to the empty processing tank 42. The third fermenting composition 41′ or the liquid product 47 may be added in an amount such that it constitute in the range of from 5 to 50% of the total volume of the processing tank 32, such as about 25 to 35% of the total volume. A second batch of the mixed soluti 43′ is thereafter added to the processing tank 42 comprising the third fermenting composition 41′ or the liquid product 47. The added second batch of mixed solution 43 may constitute the remaining volume in the processing tank 42, such as in the range of from 50 to 95% of the total volume of the transportation tank 42, such as in the range of from 65 to 75% of the total volume. The second batch of mixed solution 43 is mixed 44′ with the third fermenting composition 41′ or the liquid product 47, whereby a second fermented mixture 45′ is obtained. It may also be possible to add the second batch of mixed solution 43′ to the processing tank 42 prior to adding the third fermenting composition 41′ or the liquid product 47. The fermented mixture 45′ may subsequently be separated 46′ into a second liquid product 47′ and a second solid product 48′. Any third or further cycles of the inventive method may thereafter repeat the described method steps of adding, mixing and separating in order to produce further liquid and solid products.

The liquid product 37, 47 may be reused as the first fermenting composition 21 in the optional initial steps of the inventive method.

The solid product 38, 48 may further be wet ground, and optionally subsequently vacuum packaged. The wet ground and vacuum packaged solid product has a prolonged durability compared to the batch of spent grain 23 and the batch of mixed solution 33, 43. It may be stored for up to several months in a cooled storage space or in a refrigerator. The wet ground solid product may be used as an ingredient in the food industry, such as bakery industry, dairy industry or fast food industry. The wet ground solid product may be present as a basic ingredient at an amount of between 5 to 30% of the total weight of a food product. The food product comprising the wet ground solid product also has a prolonged durability as well as probiotic effect thanks to the inventive method.

### EXAMPLES

Below it will be described how the first fermenting composition, for use in the inventive method, was prepared. It should however be noted that also further fermenting compositions capable of fermenting a batch of spent grain and/or a mixed solution may be used.

### Example I

### Preparing a Pre-Fermenting Composition

### Aim of Example I

To provide a pre-fermenting composition.

### MATERIALS AND METHODS

1 L of a carbohydrate solution was heated to 100° C. and was allowed to boil for about 10 minutes. The carbohydrate solution used was milk. Alternatively, other types of carbohydrate solutions may also be used such as a solution of grain, e.g. rye, mixed with water, such as a mixture of 10-20% grain and 80-90% water, or a solution of monosaccharides, such as glucose, fructose or lactose or any combinations thereof, mixed with water, such as a mixture of 5-10% monosaccharides and 90-95% water. After the carbohydrate solution had boiled for about 10 minutes its temperature was lowered to about 40° C. Thereafter, lactic acid bacteria were added to the carbohydrate solution. The lactic acid bacteria being added were Lactobacillus helveticus 14492 (4 mL), Lactobaciullus kefir 14502 (2 mL), and Lactobacillus acidophilus 14499 (2 mL). The lactic acid bacteria were mixed with the carbohydrate solution and were allowed to ferment for about 24 hours at a temperature in the range of from 37-40° C., whereby a pre-fermenting composition was obtained.

### RESULTS AND CONCLUSIONS

A pre-fermenting composition was obtained.

### Example II

### Preparing Larger Batches of Pre-Fermenting Composition

### Aim of Example II

To provide larger batch of the pre-fermenting composition obtained in Example I.

### MATERIALS AND METHODS

9 L of a carbohydrate solution was heated to 100° C. and was allowed to boil for about 10 minutes. The carbohydrate solution used was a solution of grain, e.g. rye, mixed with water, such as a mixture of 10-20% grain and 80-90% water. Alternatively, a solution of monosaccharides, such as glucose, fructose or lactose or any combinations thereof, mixed with water, such as a mixture of 5-10% monosaccharides and 90-95% water may also be used. After the carbohydrate solution had boiled for about 10 minutes its temperature was lowered to about 40° C. Thereafter, the pre-fermenting composition obtained in Example I was added to the carbohydrate solution having a temperature of about 40° C. The pre-fermenting composition was mixed with the carbohydrate solution and was allowed to ferment for about 24 hours at a temperature in the range of from 37-40° C., whereby about 10 L of a first batch of pre-fermenting composition was obtained.

90 L of a carbohydrate solution was heated to 100° C. and was allowed to boil for about 10 minutes. The carbohydrate solution used was a solution of grain, e.g. rye, mixed with water, such as a mixture of 10-20% grain and 80-90% water. Alternatively, a solution of monosaccharides, such as glucose, fructose or lactose or any combinations thereof, mixed with water, such as a mixture of 5-10% monosaccharides and 90-95% water may also be used. After the carbohydrate solution had boiled for about 10 minutes its temperature was lowered to about 40° C. Thereafter, the first batch of pre-fermenting composition (about 10 L) obtained according to the above was added to the carbohydrate solution having a temperature of about 40° C. The first batch of pre-fermenting composition was mixed with the carbohydrate solution and were allowed to ferment for about 24 hours at a temperature in the range of from 37-40° C., whereby about 100 L of a second batch of pre-fermenting composition was obtained.

A third batch of pre-fermenting composition was obtained by preparing 900 L of carbohydrate solution as described above and mixed with the 100 L of second batch of pre-fermenting composition was obtained. The described procedure was repeated until a desired amount of pre-fermenting composition was obtained. The pre-fermenting composition or the further (i.e. first, second etc.) batch of pre-fermenting composition constitute about 10% of the total volume.

### RESULTS AND CONCLUSIONS

A larger batch of pre-fermenting composition was obtained.

### Example III

### Preparing a First Fermenting Composition

### Aim of Example II

To provide a first fermenting composition.

### MATERIALS AND METHODS

The larger batch of pre-fermenting composition obtained in Example II was filtered in order to remove solid components therein. After the filtration, the solid components were discharged. The obtained filtrate comprising lactic acid bacteria and enzymes may be the first fermenting composition which is used in the inventive method. The first fermenting composition had a pH in the range of from about 3.5 to 4.5. The obtained first fermenting composition may be stored in room temperature without turning rancid or in a refrigerator. The lactic acid bacteria may be latent within the first fermenting composition when stored and may be activated when they are contacted with nutrition, such as carbohydrates, and a temperature of from about 37-40° C.

### RESULTS AND CONCLUSIONS

A first fermenting composition was obtained.

1. A method for recovering spent grain, comprising the steps of:
a) adding a batch of mixed solution of a batch of spent grain and a first fermenting composition to a second fermenting composition;
b) mixing the added batch of mixed solution with the second fermenting composition, whereby a fermented mixture is obtained; and
c) separating the fermented mixture into a liquid product and a solid product;
wherein the liquid product and the solid product have a prolonged durability compared to the durability of the batch of spent grain when mixed solution has been added in step a).

2. The method according to claim 1, wherein the method prior to step a) comprises the steps of:
i. adding the batch of spent grain to the first fermenting composition; and
ii. mixing the added batch of spent grain with the first fermenting composition, whereby the mixed solution is obtained.

3. The method according to claim 1, wherein the batch of mixed solution is a first batch of mixed solution, wherein a portion of the fermented mixture obtained in step b) is reused in the method comprising the steps of:
a) adding a second batch of mixed solution to the portion of fermented mixture;
b) mixing the added second batch of mixed solution with the portion of fermented mixture, whereby a second fermented mixture is obtained; and
c) separating the second fermented mixture into a second liquid product and a second solid product.

4. The method according to claim 1, wherein the batch of mixed solution is a first batch of mixed solution, wherein the liquid product obtained in step c) is reused in the method comprising the steps of:
a) adding a third batch of mixed solution to the liquid product;
b) mixing the added third batch of mixed solution with the liquid product, whereby a third fermented mixture is obtained; and
c) separating the third fermented mixture into a third liquid product and a third solid product.

5. The method according to claim 1, wherein;
when dependent on claim 1, water is added in step a) to the second fermenting composition;
when dependent on claim 3, water is added in step a) to the portion of fermented mixture; and
when dependent on claim 4, water is added in step a) to the liquid product.

6. The method according to claim 1, wherein the method further comprises the steps of:
d) wet grinding the solid product, the second solid product and/or the third solid product, and optionally
e) vacuum packaging the wet ground solid product, the wet ground second solid product and/or the wet ground third solid product.

7. The method according to claim 6, wherein the step d) further comprises spray-drying the wet ground solid product, the wet ground second solid product and/or the wet ground third solid product.

8. The method according to claim 1, wherein the second fermenting composition, the portion of fermented mixture or the liquid product being 5 to 50% of the total volume, such as 25 to 35% of the total volume.

9. The method according to claim 2, wherein the method prior to step i) initially comprises adjusting the pH of the first fermenting composition to a pH below 4.5, such as a pH in the range of from 3.5 to 4.5.

10. The method according to claim 1, wherein the separation in step c) is performed by filtration or centrifugation.

11. The method according to claim 1, wherein the mixing in step b) is conducted at a temperature in the range of from 5 to 50° C., such as in the range of from 20 to 45° C., e.g. in the range of from 37 to 40° C.

12. The method according to claim 1, wherein the mixing in step b) is performed within a time range of from 4 to 24 hours.

13. The method according to claim 1, wherein the first and/or second fermenting composition comprising fermenting bacteria being lactic acid bacteria, such as lactic acid bacteria chosen from the group consisting of Lactobacillus helveticus 14492, Lactobaciullus kefir 14502, and Lactobacillus acidophilus 14499, or any combinations thereof.

14. A liquid product obtainable by the method according to claim 1.

15. A solid product obtainable by the method according to claim 1.

16. Use of the liquid product according to claim 14 as an ingredient in human food.

17. Use of the solid product according to claim 15 as an ingredient in human food.
